(19) United States
(12) Patent Application Publication
(10) Pub. No.: US 2024/0024518 A1
(43) Pub. Date: Jan. 25, 2024

(54) CONTRAST AGENT AND PRECURSOR AND MANUFACTURING METHOD THEREOF

(71) Applicant: INSTITUTE OF NUCLEAR ENERGY RESEARCH, ATOMIC ENERGY COUNCIL, EXECUTIVE YUAN, Taoyuan (TW)

(72) Inventors: Shiu-Wen LIU, Taoyuan (TW); Yi-Jhih CHEN, Taoyuan (TW); Wei-Hsi CHEN, Taoyuan (TW); Yu CHANG, Taoyuan (TW); Cheng-Fang HSU, Taoyuan (TW)

(73) Assignee: INSTITUTE OF NUCLEAR ENERGY RESEARCH, ATOMIC ENERGY COUNCIL, EXECUTIVE YUAN, Taoyuan (TW)

(21) Appl. No.: 18/099,188

(22) Filed: Jan. 19, 2023

(30) Foreign Application Priority Data
Jul. 19, 2022 (TW) 111126990

(51) Int. Cl. A61K 51/04

(52) U.S. Cl. CPC A61K 51/04

(57) ABSTRACT

The present invention provides a contrast agent which is efficiently and specifically absorbed by tumor cells, suitable for use in single photon emission computed tomography for the diagnosis, efficacy assessment and tumor tracking of neuroblastoma, pheochromocytoma or congestive heart failure.

### FIELD OF THE INVENTION

The present invention relates to a contrast agent, and more particularly relates to a contrast agent, a precursor thereof manufactured using m-Iodobenzylguanidine hydrosulfate (MIBGHS) and a manufacturing method of the precursor.

### BACKGROUND OF THE INVENTION

Neuroblastoma is a cancer that develops from neuroblasts, which usually occurs in infants and young children. Neuroblastoma usually develops in the nervous system, except for the brain, before birth and may metastasize to bone or bone marrow.

The exact cause of the disease is still uncertain. Most neuroblastomas are not hereditary, and only about 1% to 2% of children with neuroblastoma have a family history, so in other cases, the cause remains uncertain.

Neuroblastoma is one of the most common solid tumors in children, accounting for 8%-10% of all childhood tumors and 15% of all cancer-related deaths in the pediatric population. Based on available observations, the 5-year survival rate for this disease is approximately 40-50%.

Since the discovery in the 1980s that the 1-123 and 1-131 labeled products (123 I-MIBG and 131 I-MIBG) of 3-iodobenzylguanidine (MIBG) are analogs of norepinephrine and can be widely used as nuclear medicine contrast agents for heart disease, pheochromocytoma and neuroblastoma, MIBG has now become the main contrast agent for neuroblastoma imaging worldwide.

However, domestic 131 I-MIBG is very dependent on imports, and manufacturers spend a lot of manpower and material resources in applying for licenses, thus reducing manufacturers' willingness to import, resulting in the current domestic predicament of having no medicine available except for applications for project import.

There are also documents that recorded the synthetic method of MIBG. In 1973, Cymerman et al. proposed a synthetic method to obtain MIBG by treating guanidine hydrochloride (Guanidine HCl) with sodium ethoxide, adding m-iodobenzyl bromide after the sodium chloride was filtered, and then performing methanol crystallization after the reaction was completed. The yield of this method is only 43%, and since sodium metal is required in the process, the risk is high, and a relatively stable salt product cannot be obtained, which may affect the stability of the Active Pharmaceutical Ingredient (API).

In 1923, Lecher et al. published a synthesis method of MIBG, in which 5-methoxy-2-iodophenyl borate hydrochloride (MIBA HCl) and cyanoguanidine are mixed, heated to 200° C. and refluxed under a nitrogen atmosphere for 6 hours, and after the reaction was completed, cooled to room temperature to obtain MIBG. This method only simply mixes the two starting reagents without co-dissolved in any reagents, so the possible by-products cannot be effectively separated, and a more stable form of salt products cannot be obtained, which may affect the stability of the API.

In 2013, Sheikholislam et al. published a synthetic method of MIBG, in which MIBA HCl and s-Ethylisothiouronium sulfate are mixed in water, stirred at room temperature, and after the reaction was completed, the water is first removed and the resulting crude product is recrystallized with acetone to obtain stable hemisulfate product MIBG Hemisulfate (MIBGHS). However, this method has a high proportion of ethanethiol in the product, and s-ethylisothiourea sulfate is not commercially available, so additional synthesis steps are required.

In 2004, Valliant et al. published a synthesis method of MIBG, in which MIBA HCl and Aminoiminomethanesulfonic acid are mixed in methanol, heated and refluxed for 16 to 24 hours, and after the reaction was completed, the methanol is drained to obtain the sulfite product of MIBG. However, this method has a high proportion of by-products in the product, aminoimine methanesulfonic acid is not commercially available, so additional synthesis steps are required, and the reaction time is longer and a more stable form of salt products cannot be obtained, which may affect the stability of the API.

In 2016, CHEN ZHIMING et al. published a synthetic method of MIBG, in which m-iodobenzylamine hydrochloride and cyanamide are mixed directly, heated to 100° C. and reacted for 8 hours, cooled by adding water, and then added potassium hydrogen (KHCO 3) to obtain a first step product, and then the first step product is added into water, dropped with 1N H₂SO₄ sulfuric acid, and the resulting crude product is recrystallized with ethanol aqueous solution to obtain MIBG. The reaction time is long and a considerable proportion of by-products are produced, and the purity is low.

In 2014, Stephen et al. published a synthetic method of MIBG, in which m-iodobenzylguanidine hydrochloride and cyanamide are mixed, heated and refluxed at 100-110° C. overnight, cooled and dissolved in water, then dropped with NaHCO₃ aqueous solution to precipitate the solid, and then subjected to a suction filtration to obtain bicarbonate MIBG product. However, this method has the disadvantages that the reaction time is long, there are many by-products, the product is bicarbonate MIBG, and a more stable form of the hemisulphate product cannot be obtained, which may affect the stability of the crude drug.

Therefore, there is a need for a manufacturing method of contrast agent that is simple, has short reaction time, high yield, effectively separation of the by-product, and produces a high-purity and stable form of salt product.

### SUMMARY OF THE INVENTION

In order to achieve the above objective, the present invention provides a contrast agent precursor represented by the following chemical formula (1):

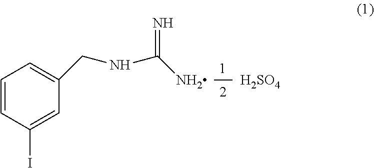

In one embodiment, the present invention provides a contrast agent formed by substituting a radioactive isotope iodine-123 as a marker on the contrast agent precursor as described above.

In one embodiment, the present invention provides a contrast agent formed by substituting a radioactive isotope iodine-131 as a marker on the contrast agent precursor as described above.

According to one embodiment of the present invention, the contrast agent is provided for diagnosing neuroblastoma.

According to one embodiment of the present invention, the contrast agent is provided for diagnosing pheochromocytoma.

According to one embodiment of the present invention, the contrast agent is provided for diagnosing congestive heart failure.

According to one embodiment of the present invention, the contrast agent is a contrast agent for single photon computed tomography.

According to one embodiment, the present invention provides a method of manufacturing the contrast agent precursor above, comprising: a first step: heating a first mixture which is obtained by mixing 8.02 g of m-iodobenzylamine hydrochloride and 2.5 g of cyanamide at 105° C. for 4 hours to make the first mixture appear as a transparent oil, after cooling the first mixture, stirring and dissolving 15 mL of water into the first mixture, dropping the first mixture into 30 mL of an aqueous solution in which 2.50 g of sodium bicarbonate is dissolved to precipitate a white solid in the first mixture, and stirring for 15 minutes; a second step: performing suction filtration on the first mixture with white solid precipitated to obtain a first filtration product, washing the first filtration product with cold water to obtain a first precipitated solid, placing the first precipitated solid in 30 mL of water to obtain a first solution, adding 2N H2SO4 aqueous solution dropwise to adjust the first solution to pH 2 in an ice bath state, heating the first solution to 80° C. to completely dissolve the first precipitated solid, then cooling the first solution to precipitate solids in the first solution, performing suction filtration on the first solution and wash the product of suction filtration with cold water to obtain a second precipitated solid; and a third step: mixing the second precipitated solid with a 1:1 aqueous ethanol solution to obtain a first aqueous mixture, heating the first aqueous mixture slowly to 60° C. to completely dissolve the second precipitated solid, cooling the first aqueous mixture to room temperature, further cooling the first aqueous mixture with refrigerator, adding 2N H2SO4 aqueous solution to adjust the first aqueous mixture to pH2, cooling the first aqueous mixture with refrigerator again, filtering the first aqueous mixture, drying the first aqueous mixture to obtain a third precipitated solid, and washing the third precipitated solid with chloroform to obtain the contrast agent precursor.

With the technical means adopted by the present invention, the present invention can provide a contrast agent that is effective absorbed by tumor cells and suitable for use in single-photon computed tomography, and m-iodobenzylguanidine hydrosulfate (MIBGHS) as a precursor thereof. The contrast agent is used to diagnose, evaluate curative effect and track tumor recurrence of neuroblastoma, pheochromocytoma, and congestive heart failure. Moreover, the present invention can obtain MIBGHS with high purity in simple steps, and inorganic impurities and various solvent residues in MIBGHS are in compliance with the API license Good Manufacturing Practice (GMP).

### DETAILED DESCRIPTION OF THE PREFERRED EMBODIMENTS

The preferred embodiments of the present invention are described in detail below. The description is for explaining the embodiments of the present invention only, but not for limiting the scope of the claims.

An embodiment of the present invention is described with reference to formula (1), in which m-Iodobenzylguanidine hydrosulfate (MIBGHS) is used as a precursor of a contrast agent.

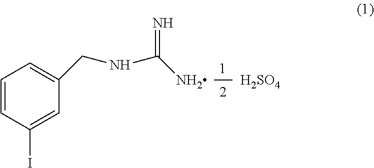

The MIBGHS of the present invention is a hemi-sulfate product of MIBG, the main contrast agent for neuroblastoma imaging, which is more stable salt than MIBG while being effectively and specifically absorbed by neuroblastoma.

The MIBGHS-based contrast agent precursor of the present invention can be effectively coupled with 1-123 or 1-131 for the detection of the primary lesion or metastasis of pheochromocytoma or neuroblastoma, and for the adjunct use of other diagnostic tests; for the assessment of myocardial sympathetic nerve function by calculating the ratio of radioactivity uptake in the heart and the mediastinum; and for the differential diagnosis of degenerative brain diseases.

According to another embodiment of the present invention, the contrast agent is formed by substituting a radioisotope 1-123 as a marker on the contrast agent precursor as described above.

The I-123-MIBGHS of the present invention can provide more photon counts than the prior art, thereby improving image quality, and can be used in single photon emission computed tomography (SPECT) technology to capture images, and have positive contribution to the certainty and localization of tumor diagnosis. I-123-MIBGHS is also lower than I-131-MIBGHS in terms of radiation dose. Further, because 1-123 is pure gamma photon rays with a photoelectric peak of 159 keV, it is more suitable for imaging than 1-131 which emits both gamma photons and beta rays with high-energy peaks.

According to another embodiment of the present invention, the contrast agent above is a contrast agent for diagnosing neuroblastoma.

According to another embodiment of the present invention, the contrast agent above is a contrast agent for diagnosing pheochromocytoma.

According to another embodiment of the present invention, the contrast agent above is a contrast agent for diagnosing congestive heart failure.

The contrast agent of the present invention marking tumor cells with 1-123 by using the characteristic that the tumor cell membrane of neuroblastomas such as pheochromocytoma, neuroendocrine tumor and medullary thyroid carcinoma can actively absorb MIBGHS and store it in cytoplasmic secretory vesicles. 1-123 is a highly specific iodine label which can bind to adrenal receptors. It has been clinically recognized as an effective diagnostic and localization function for neuron-derived tumor diseases, and the imaging intensity can be use for evaluation of the therapeutic effect of internal metastases in tumor and bone marrow.

According to another embodiment of the present invention, the contrast agent above is a contrast agent for single photon computed tomography.

According to the present invention, the contrast agent can be effectively and specifically absorbed by tumor cells, and is suitable for single-photon computed tomography scanning for neuroblastoma diagnosis, therapeutic evaluation and tracking of tumor recurrence.

According to an embodiment of the present invention, the method of manufacturing the contrast agent precursor above comprises:
a first step:
heating a first mixture which is obtained by mixing 8.02 g of m-iodobenzylamine hydrochloride and 2.5 g of cyanamide at 105° C. for 4 hours to make the first mixture appear as a transparent oil,
after cooling the first mixture, stirring and dissolving 15 mL of water into the first mixture, dropping the first mixture into 30 mL of an aqueous solution in which 2.50 g of sodium bicarbonate is dissolved to precipitate a white solid in the first mixture, and stirring for 15 minutes.

Controlling the temperature at 105° C. can stabilize the preliminary reaction of meta-iodobenzylamine hydrochloride and cyanamide, and avoid the by-product caused by the conversion of cyanamide to dicyandiamide. In addition, by making the molar ratio of 1:2:1 between m-iodobenzylamine hydrochloride, cyanamide and an alkaline substance, namely sodium bicarbonate aqueous solution, the reaction can be made more complete, and the residual raw materials can be minimized. Further, using sodium bicarbonate as an alkaline substance has the characteristics of low cost, low environmental pollution, and no skin irritation.

A second step:
performing suction filtration on the first mixture with white solid precipitated to obtain a first filtration product, washing the first filtration product with cold water to obtain a first precipitated solid, placing the first precipitated solid in 30 mL of water to obtain a first solution, adding 2N H2SO4 aqueous solution dropwise to adjust the first solution to pH 2 in an ice bath state, heating the first solution to 80° C. to completely dissolve the first precipitated solid, then cooling the first solution to precipitate solids in the first solution, performing suction filtration on the first solution and wash the product of suction filtration with cold water to obtain a second precipitated solid.

In the step of dropping the H₂SO₄ solution of 2N, it is preferable to slowly drop the 2N H₂SO₄ aqueous solution, measure the pH value of the first solution immediately, and stop the reaction when the pH value reaches the specified value to prevent by-products.

And, a third step:
mixing the second precipitated solid with a 1:1 aqueous ethanol solution to obtain a first aqueous mixture, heating the first aqueous mixture slowly to 60° C. to completely dissolve the second precipitated solid, cooling the first aqueous mixture to room temperature, further cooling the first aqueous mixture with refrigerator, adding 2N H2SO4 aqueous solution to adjust the first aqueous mixture to pH2, cooling the first aqueous mixture with refrigerator again, filtering the first aqueous mixture, drying the first aqueous mixture to obtain a third precipitated solid, and washing the third precipitated solid with chloroform to obtain the contrast agent precursor.

The manufacturing method of the contrast agent precursor of the present invention has the following advantages over the prior art: higher yield, relatively simple separation of by-products, shorter reaction time, stable salt product, good stability of the raw material drug, and inorganic impurities and various solvent residues in compliance with the Good Manufacturing Practices. The manufacturing method of the present invention is simple and convenient to operate, easy to scale up, and has high yield and high purity.

What is claimed is:

1. A contrast agent precursor represented by the following general formula (1):

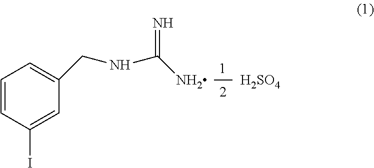

2. A contrast agent formed by substituting a radioactive isotope iodine-123 as a marker on the contrast agent precursor as claimed in claim 1.

3. A contrast agent formed by substituting a radioactive isotope iodine-131 as a marker on the contrast agent precursor as claimed in claim 1.

4. The contrast agent as claimed in claim 2, wherein the contrast agent is a contrast agent for diagnosing neuroblastoma.

5. The contrast agent as claimed in claim 3, wherein the contrast agent is a contrast agent for diagnosing neuroblastoma.

6. The contrast agent as claimed in claim 2, wherein the contrast agent is a contrast agent for diagnosing pheochromocytoma.

7. The contrast agent as claimed in claim 3, wherein the contrast agent is a contrast agent for diagnosing pheochromocytoma.

8. The contrast agent as claimed in claim 2, wherein the contrast agent is a contrast agent for diagnosing congestive heart failure.

9. The contrast agent as claimed in claim 3, wherein the contrast agent is a contrast agent for diagnosing congestive heart failure.

10. The contrast agent as claimed in claim 2, wherein the contrast agent is a contrast agent for single photon computed tomography.

11. The contrast agent as claimed in claim 3, wherein the contrast agent is a contrast agent for single photon computed tomography.

12. A method of manufacturing the contrast agent precursor as claimed in claim 1, comprising:
a first step:
heating a first mixture which is obtained by mixing 8.02 g of m-iodobenzylamine hydrochloride and 2.5 g of cyanamide at 105° C. for 4 hours to make the first mixture appear as a transparent oil,
after cooling the first mixture, stirring and dissolving 15 mL of water into the first mixture, dropping the first mixture into 30 mL of an aqueous solution in which 2.50 g of sodium bicarbonate is dissolved to precipitate a white solid in the first mixture, and stirring for 15 minutes;
a second step: performing suction filtration on the first mixture with white solid precipitated to obtain a first filtration product, washing the first filtration product with cold water to obtain a first precipitated solid, placing the first precipitated solid in 30 mL of water to obtain a first solution, adding 2N H2SO4 aqueous solution dropwise to adjust the first solution to pH 2 in an ice bath state, heating the first solution to 80° C. to completely dissolve the first precipitated solid, then cooling the first solution to precipitate solids in the first solution, performing suction filtration on the first solution and wash the product of suction filtration with cold water to obtain a second precipitated solid; and
a third step: mixing the second precipitated solid with a 1:1 aqueous ethanol solution to obtain a first aqueous mixture, heating the first aqueous mixture slowly to 60° C. to completely dissolve the second precipitated solid, cooling the first aqueous mixture to room temperature, further cooling the first aqueous mixture with refrigerator, adding 2N H2SO4 aqueous solution to adjust the first aqueous mixture to pH2, cooling the first aqueous mixture with refrigerator again, filtering the first aqueous mixture, drying the first aqueous mixture to obtain a third precipitated solid, and washing the third precipitated solid with chloroform to obtain the contrast agent precursor.
